(19) United States
(12) Patent Application Publication
(10) Pub. No.: US 2023/0313140 A1
(43) Pub. Date: Oct. 5, 2023

(54) METHOD FOR PREPARING T CELLS FOR ADOPTIVE T CELL THERAPY

(71) Applicant: St. Jude Children's Research Hospital, Inc., Memphis, TN (US)

(72) Inventors: Swantje LIEDMANN, Memphis, TN (US); Ao GUO, Memphis, TN (US); Douglas R. GREEN, Memphis, TN (US)

(21) Appl. No.: 17/712,787

(22) Filed: Apr. 4, 2022

(51) Int. Cl. C12N 5/0783; A61K 35/17; A61P 35/00

(52) U.S. Cl. CPC C12N 5/0638; A61K 35/17; A61P 35/00; C12N 2501/73; C12N 2510/00; C12N 2501/515

(57) ABSTRACT

Disclosed is a method for preparing T cells for adoptive T cell therapy by contacting a population of activated T cells with an Eukaryotic initiation factor-4A (eIF4A) inhibitor. Also disclosed is a kit, a population of T cells or engineered T cells produced by the method and use of the same in adoptive T cell therapy and the treatment of cancer.

### INTRODUCTION

This invention was made with government support under grant nos. AI123322, AI136514 and AI154470 awarded by the National Institutes of Health. The government has certain rights in this invention.

### BACKGROUND

Chimeric antigen receptor (CAR)-T cell therapy has changed the landscape of treatment options for B cell malignancies. However, frequent relapses in treated patients, together with inability to achieve complete remission in certain disease types, highlight the need of further potentiating this therapeutic strategy.

Extensive clinical experience has indicated that primary objective responses are associated with the level of CAR-T cell expansion early after infusion, while long-term persistence is required to prevent relapses. Among others, intrinsic T cell properties and composition of the infused T cell product have been reported to significantly shape CAR-T cell fitness. T cells exist in a wide range of interconnected differentiation statuses, differing in terms of proliferative capacity, self-renewal capabilities and long-term survival. In this regard, evidence in mice and humans suggests that T cell differentiation negatively correlates with long-term antitumor activity, with early memory T cells holding the most favorable features. Accordingly, T cells from chronic lymphocytic leukemia patients who responded to CD19 CAR-T cells were found enriched in gene expression profiles involved in early memory, or were rather the result of a single central memory T-cell (T_{CM}) clone deriving from a TET2-targeted insertional mutagenesis event (Majzner & Mackall (2019) Nat. Med. 25:1341-1355; Fraietta et al. (2018) Nat. Med. 24:563-571; Fraietta et al. (2018) Nature 558:307-312).

Therefore, there is a need in the art for generating T cells with memory potential or function that persist and provide improved therapeutic benefits.

### SUMMARY OF THE INVENTION

This invention is based on the discovery that treatment of naïve T cells with an inhibitor of Eukaryotic initiation factor-4A (eIF4A) during activation, and prior to adoptive T cell therapy promotes memory-like potential of these cells and increases sternness and longevity. Accordingly, this invention provides a method for preparing T cells for adoptive T cell therapy by contacting a population of activated T cells, e.g., CD8⁺ T cells, with an eIF4A inhibitor. In some aspects, activation of the population of T cells is via stimulation of CD3, CD28, or a combination thereof. In other aspects, the method further includes the step of expanding the activated population of T cells with one or more cytokines. In further aspects, the method includes the step of introducing into said population of T cells an exogenous nucleic acid molecule, e.g., an antigen recognizing receptor (e.g., a T cell receptor (TCR) or a chimeric antigen receptor (CAR)), an ortho-receptor, an immunomodulatory cytokine, a chemokine receptor, a dominant-negative receptor, or a transcription factor for preventing exhaustion, thereby producing a population of engineered T cells. Antigens of use in this aspect include, e.g., a tumor antigen, a self-antigen, or a pathogen antigen. Populations of T cells or engineered T cells prepared by the methods of this invention, and pharmaceutical compositions containing the same, are also provided, as are methods for using the T cells for adoptive T cell therapy and treating cancer. This invention further provides a kit for preparing T cells for adoptive T cell therapy, which includes (a) an eIF4A inhibitor, (b) CD3 agonist, and optionally (c) a costimulatory ligand and/or (d) one or more cytokines.

### BRIEF DESCRIPTION OF THE DRAWINGS

FIG. 1 shows the Median Fluorescence Intensity (MFI) of GFP-c-Myc by fluorescence-activated cell sorting (FACS) analysis. CD8⁺ T cells were activated for 36 hours on peptide-pulsed antigen presenting cells (APCs), treated with silvestrol (Silv) for 2 hours, washed and cultured for the indicated time; mean±SD, relative to CD8^{high} control (ctrl). Data compiled from two independent experiments.

FIG. 2 shows the frequency of donor cells on day 9 after A/PR/8-OVA challenge. First-division c-Myc^{high} or c-Myc^{low} cells, treated with silvestrol for 2 hours after sorting, were adoptively transferred into recipient animals before infection with A/X-31-OVA (day 0) and challenge with A/PR/8-OVA (day 30). Each dot represents a recipient animal, n=10; mean±SD. Significance was calculated with ANOVA; *p<0.05, ns=not statistically significant.

### DETAILED DESCRIPTION OF THE INVENTION

It has now been found that transient treatment of activated T cells with an eIF4A inhibitor promotes memory-like potential and enhances persistence and longevity in vivo compared to conventionally activated T cells. Notably, transient inhibition of eIF4A during preparation of T cells, in particular at approximately the time of the first division, reduces c-Myc levels in the period following first division and is sufficient to alter T cell fate trajectories. The ability to shape T cell function through minimal pharmacological intervention provides therapeutic avenues to improve vaccines and advance T cell immune therapies.

According, the present invention provides a method for preparing T cells for adoptive T cell therapy by contacting a population of activated T cells with an eIF4A inhibitor. The terms “T cell” or “T lymphocyte” are art-recognized and are intended to include thymocytes, naïve T lymphocytes, immature T lymphocytes, mature T lymphocytes, resting T lymphocytes, or activated T lymphocytes. Illustrative populations of T cells suitable for use in the methods of this invention include but are not limited to helper T cells (HTL; CD4⁺ T cells), cytotoxic T cells (CTL; CD8⁺ T cells), CD4⁺ CD8⁺ T cells, or any other suitable subset of T cells. Other illustrative populations of T cells suitable for use include T cells expressing one or more of the following markers: CD3, CD4, CD8, CD27, CD28, CD45RA, CD45RO, CD62L, CD127, CD197, and HLA-DR. In particular aspects, the population of T cells comprises, consists essentially of, consists of, or is composed substantially of (e.g., more than 90%, 95%, 97%, 98%, 99%) CD8⁺ T cells.

T cells for adoptive T cell therapy may be autologous/autogeneic (“self”) or non-autologous (“non-self,” e.g., allogeneic, syngeneic, or xenogeneic). “Autologous” refers to cells from the same subject. “Allogeneic” refers to cells of the same species that differ genetically to the cell in comparison. “Syngeneic” refers to cells of a different subject that are genetically identical to the cell in comparison. “Xenogeneic” refers to cells of a different species to the cell in comparison.

In one aspect, the T cells are obtained from a mammalian subject. In another aspect, the cells are obtained from a primate subject. In a particular aspect, the cells are obtained from a human subject. The population of T cells can be obtained from a number of sources including, but not limited to, peripheral blood, peripheral blood mononuclear cells, bone marrow, lymph nodes tissue, cord blood, thymus issue, tissue from a site of infection, ascites, pleural effusion, spleen tissue, and tumors. In certain aspects, the population of T cells are isolated from the circulating blood of an individual by apheresis, e.g., leukapheresis. The apheresis product may contain lymphocytes, including T cells, monocytes, granulocytes, B cells, other nucleated white blood cells, red blood cells, and platelets or may be a leukapheresis product including lymphocytes, including T cells, monocytes, granulocytes, B cells, and other nucleated white blood cells. The cells collected by apheresis may be washed to remove the plasma fraction and to place the cells in an appropriate buffer or media for subsequent processing. The cells can be washed with phosphate-buffered saline (PBS) or with another suitable solution that lacks calcium, magnesium, and most, if not all other, divalent cations.

Once a population of cells containing T cells has been obtained, cell counts and viability of cells within the population of cells can be determined, the population or portions thereof may be cryopreserved for future use or analyses, and cells in the population, e.g., PBMCs, may be characterized using a number of cell marker panels, e.g., CD3, CD4, CD8, CD14, CD16, CD19, CD28, CD45RA, CD45R0, CD61, CD62L, CD66b, CD127, and HLA-DR, and maintained in T cell culture medium.

In particular aspects, a population of PBMCs is used to isolate a population of T cells. Specific cell types can be isolated from PBMCs as described herein or by conventional methods. In some aspects, cytotoxic and helper T lymphocytes can be sorted into naïve, memory, and effector T cell subpopulations either before or after activation, expansion, and/or genetic modification. As an alternative, T cells may be obtained commercially, e.g., Sanguine Biosciences.

Once isolated, the population of T cells is activated. The terms “activated” or “activation” refer to the state of a T cell that has been sufficiently stimulated to induce detectable cellular proliferation. In some aspects, activation can also be associated with induced cytokine production. The term “activated T cells” refers to, among other things, T cells that are proliferating. As is conventional in the art, “stimulation” refers to a primary response induced by binding of a stimulatory molecule with its cognate ligand thereby mediating a signal transduction event including, but not limited to, signal transduction via the TCR/CD3 complex or via stimulation of the CD2 surface protein. Preferably, the population of T cells of this invention are activated by stimulating CD3. In some aspects, stimulation of CD3 is carried out with a CD3 agonist. A suitable CD3 agonist includes aa CD3 ligand or anti-CD3 antibody, in particular an activating antibody. Illustrative examples of CD3 antibodies include, but are not limited to, OKT3, G19-4, BC3, and 64.1.

Signals generated through the TCR alone are often insufficient for full activation of T cells and one or more secondary or costimulatory signals may be required. Thus, T cell activation may include the use of a primary stimulation signal through the TCR/CD3 complex and one or more secondary costimulatory signals. A costimulatory signal can be achieved using a costimulatory ligand including, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor or a ligand that specifically binds with B7-H3. A costimulatory ligand also encompasses, inter alia, an antibody or antigen binding fragment thereof that specifically binds with a costimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83. In certain aspects, the T cell is activated by costimulation of CD28, e.g., with a CD28 ligand or anti-CD28 antibody. Illustrative examples of suitable anti-CD28 antibodies include monoclonal antibodies 9.3, B-T3, XR-CD28, KOLT-2, 15E8, 248.23.2, and EX5.3D10. The stimulation of CD3 and optionally a costimulatory molecule such as CD28 may be performed according to any known method in the art for instance beads, matrix, or cell-free matrix. Alternatively, aAPCs expressing anti-CD3 and anti-CD28 single chain variable fragments (scFvs) may be used (Shrestha et al. (2020) J. Immunother. 43(3):79-88).

To promote memory function of a population of activated T cells, this invention provides for contacting the population of activated T cells with an eIF4A inhibitor. An “eIF4A inhibitor,” as used herein, refers to an agent or compound that transiently blocks, inactivates, reduces, or minimizes the expression or activity of eIF4A, either alone or in a complex by about 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more as compared to untreated eIF4A. In certain aspects, an eIF4A inhibitor is a catalytic inhibitor that directly inhibits eIF4A helicase activity. An example of an eIF4A catalytic inhibitor is BPSL1549, a bacterial toxin from Burkholderia pseudomallei that deamidates Gln339 of eIF4A and converts it into a dominant-negative mutant.

In some aspects, an eIF4A inhibitor is an allosteric inhibitor. An allosteric eIF4A inhibitor binds to eIF4A at a site other than the active site, wherein its binding induces a conformational change in eIF4A so that a substrate can no longer bind eIF4A or eIF4A activity is reduced. In certain aspects, an allosteric eIF4A inhibitor includes hippuristanol and derivatives or analogs thereof. Hippuristanol, which binds the C-terminal domain of both free eIF4A (eIF4Af) and eIF4A bound in an eIF4F complex (eIF4Ac), inhibits eIF4A helicase and ATPase activities.

In some aspects, an eIF4A inhibitor is a chemical inducer of dimerization. An eIF4A chemical inducer of dimerization causes a non-sequence-specific interaction between eIF4Af and RNA and stimulates the ATP hydrolysis activity of eIF4A, resulting in sequestering of eIF4Af and depletion of eIF4Ac. Examples of eIF4A inhibitors that are chemical inducers of dimerization include pateamine A, and analogs, derivatives, or precursors thereof. Examples of pateamine A derivatives have been described in, for example, U.S. Pat. No. 7,230,021; WO 2016/161168 (α-amino derivatives that lack the C5-methyl group); and U.S. Pat. No. 7,737,134 (desmethyl, desamino-pateamine A derivatives).

In some aspects, an eIF4A inhibitor is a site-directed eIF4A inhibitor. A “site-directed eIF4A inhibitor”, as used herein, is an agent or compound that interacts with a specific nucleotide sequence of a mRNA molecule, such as a non-coding nucleotide sequence (e.g., located in the 5′-UTR of a target mRNA), and is capable of forming a stable ternary complex including the site-directed eIF4A inhibitor, an eIF4A, and a target mRNA. Exemplary site-directed eIF4A inhibitors include silvestrol, and rocaglamide compounds such as rocaglamide A, as well as analogs, derivatives, or precursors thereof. Further examples of site-directed eIF4A inhibitors include compounds as disclosed in WO 2017/091585 A1.

Representative silvestrol derivatives and analogs include CR-1-31-B, hydroxamate derivative of silvestrol, episilvestrol, silvestrol dioxane, episilvesterol dioxane, flavagline 61, (−)-4-desmethoxyepisilvestrol, and 1-O-formylaglafoline (FA). Examples of rocaglates and precursors include aglapervirisin A and aglapervirisins B-J. Further examples of silvestrol and rocaglamide derivatives and analogs are described in, for example, US 2014/0255432.

In accordance with the present disclosure, another class of example eIF4A inhibitors is cardiac glycosides. Cardiac glycosides directly bind to eIF4A and inhibit its activity (WO 2020/172086 A1). Non-limiting example cardiac glycosides include digoxin, digoxigenin, digitoxigenin, and lanotoside C.

In addition to small molecule inhibitors, eIF4A activity or expression can be reduced or inhibited using protein/peptide-based inhibitors or inhibitory RNA (IRNA) molecules (e.g., siRNA or shRNA) that are transiently expressed to disrupt eIF4A activity or expression. Exemplary IRNA constructs targeting eIF4A include, e.g., Ambion SILENCER® Select siRNA targeting eIF4A1 (s4567) an eIF4A2 (s4572); siGENOME SMARTpool siRNA targeting eIF4A1 (M-020178-01) or eIF4A2 (M-013758-01)(Dharmacon); or sheIF4A1.969 shRNA targeting eIF4A1 (Chan et al. (2019) Nat. Commun. 10:5151).

As demonstrated herein, transient contact of a population of T cells, in particular CD8⁺ T cells, promoted memory cell potential. As used herein, “transient” refers to lasting only for a brief time, e.g., less than 8 to 10 days. Notably, treatment of T cells for as short as two hours was sufficient to reduce c-Myc expression levels and alter this cell fate trajectories. Accordingly, in certain aspects of this invention, the population of activated T cells, e.g., CD8⁺ T cells or CD4⁺ T cells, is contacted with the eIF4A inhibitor immediately after activation and up to 48 hours, 72 hours, 96 hours, 102 hours, 168 hours, or 192 hours after activation. In particular aspects, the population of activated T cells is contacted with the eIF4A inhibitor immediately during the first 2, 4, 6, 8, 10, 12, 24, or 48 hours after activation, i.e., during the period between 0 hours and 48 hours after activation. Subsequently, the eIF4A inhibitor is removed, e.g., by washing to cells.

In some aspects of the invention, the method of the invention further includes expanding the activated population of T cells in one or more cytokines. Ideally, the T cells are expanded during and/or after activation and/or during and/or after contact of the T cells with the eIF4A inhibitor. Exemplary cytokines of use in expanding the activated population of T cells include, but are not limited to, IL-2, IL-15, IL-7, IL-9, IL-21, IL-23, or a combination thereof.

In certain aspects, the method further includes the step of introducing into the T cells of said population of T cells an exogenous nucleic acid molecule thereby producing an engineered T cell. As used herein, the term “engineered T cell,” “genetically engineered T cell” or “genetically modified T cell” refers to the addition of extra genetic material in the form of DNA or RNA into the total genetic material of the T cell. Preferably said introduction is performed before the expansion of the cells. Preferably the exogenous nucleic acid molecule encodes an antigen-recognizing receptor, an ortho-receptor, an immunomodulatory cytokine, a chemokine receptor, a dominant-negative receptor (for instance PD1 DDR; see Cherkassky et al. (2016) J. Clin. Invest. 126(8):3130-44), a transcription factor for preventing exhaustion (such as c-june; see Lynn et al. (2019) Nature 576(7786):293-300). In certain aspects, the antigen is a tumor antigen, a self-antigen, or a pathogen antigen. Preferably said antigen recognizing receptor is a T cell receptor (TCR) or a chimeric antigen receptor (CAR).

A TCR is a molecule which can be found on the surface of T cells that is responsible for recognizing antigens bound to MHC molecules. The naturally occurring TCR heterodimer is composed of an alpha (α) and beta (β) chain in approximately 95% of T cells, whereas about 5% of T cells have TCRs composed of gamma (γ) and delta (δ) chains. Engagement of a TCR with antigen and MHC results in activation of the T lymphocyte on which the TCR is expressed. Each chain of a natural TCR is a member of the immunoglobulin superfamily and possesses one N-terminal immunoglobulin Ig-variable (V) domain, one Ig-constant (C) domain, a transmembrane/cell membrane-spanning region, and a short cytoplasmic tail at the C-terminal end. The variable domain of both the TCR α chain or β chain have three hypervariable or complementarity determining regions (CDRs). A constant domain of a TCR may be composed of short connecting sequences in which a cysteine residue forms a disulfide bond, making a link between the two chains. An α chain of a TCR of the present invention may have a constant domain encoded by a TRAC gene. A β chain of a TCR of the present invention may have a constant domain encoded by a TRBC1 or a TRBC2 gene.

A CAR is an engineered receptor, which can confer an antigen specificity onto cells (for example T cells). CARs are also known as artificial T cell receptors, chimeric T cell receptors or chimeric immunoreceptors. Preferably the CARs of the invention include an antigen-specific targeting region, an extracellular domain, a transmembrane domain, optionally one or more co-stimulatory domains, and an intracellular signaling domain. The antigen-specific targeting domain provides the CAR with the ability to bind to the target antigen of interest. The antigen-specific targeting domain preferably targets an antigen of clinical interest against which it would be desirable to trigger an effector immune response that results in tumor killing. The antigen-specific targeting domain may be any protein or peptide that possesses the ability to specifically recognize and bind to a biological molecule (e.g., a cell surface receptor or tumor protein, or a component thereof). The antigen-specific targeting domain includes any naturally occurring, synthetic, semi-synthetic, or recombinantly produced binding partner for a biological molecule of interest. Illustrative antigen-specific targeting domains include antibodies or antibody fragments or derivatives, extracellular domains of receptors, ligands for cell surface molecules/receptors, or receptor binding domains thereof, and tumor binding proteins.

Examples of antigens which may be targeted by the CAR of the invention include but are not limited to antigens expressed on cancer cells and antigens expressed on cells associated with various hematologic diseases, autoimmune diseases, inflammatory diseases, and infectious diseases. With respect to targeting domains that target cancer antigens, the selection of the targeting domain will depend on the type of cancer to be treated. Examples of antigens specific for cancer, which may be targeted by a CAR, include but are not limited to any one or more of mesothelin, EGFRvIII, TSHR, CD19, CD123, CD22, CD30, CD171, CS-1, CLL-1, CD33, GD2, GD3, BCMA, Tn Ag, prostate specific membrane antigen (PSMA), ROR1, FLT3, FAP, TAG72, CD38, CD44v6, CEA, EPCAM, B7H3, KIT, IL-13Ra2, interleukin-11 receptor a (IL-11Ra), PSCA, PRSS21, VEGFR2, Lewis-Y, CD24, platelet-derived growth factor receptor-beta (PDGFR-beta), SSEA-4, CD20, Folate receptor α (FRα), ERBB2 (Her2/neu), MUC1, epidermal growth factor receptor (EGFR), NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-A1, legumain, HPV E6, E7, MAGE Al, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1. Preferably, the antigen-specific binding domain specifically binds to a tumor antigen.

Antigens specific for inflammatory diseases which may be targeted by the CAR of the invention include but are not limited to any one or more of AOC3 (VAP-1), CAM-3001, CCL11 (eotaxin-1), CD125, CD147 (basigin), CD154 (CD40L), CD2, CD20, CD23 (IgE receptor), CD25 (α chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11a), MEDI-528, myostatin, OX-40, rhuMAbb7, scleroscin, SOST, TGF-β, TNF-α or VEGF-A.

Antigens specific for neuronal disorders which may be targeted by the CAR of the invention include but are not limited to any one or more of beta amyloid or MABT5102A.

Antigens specific for cardiovascular diseases which may be targeted by the CARs of the invention include but are not limited to any one or more of C5, cardiac myosin, CD41 (integrin alpha-IIb), fibrin II, beta chain, ITGB2 (CD18) and sphingosine-1-phosphate.

The CAR also includes one or more co-stimulatory domains. This domain may enhance cell proliferation, cell survival and development of memory cells. Each co-stimulatory domain includes the co-stimulatory domain of any one or more of, for example, a MHC class I molecule, a TNF receptor protein, an immunoglobulin-like protein, a cytokine receptor, an integrin, a signaling lymphocytic activation molecule (SLAM protein), an activating NK cell receptor, BTLA, a Toll ligand receptor, 0X40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (CD150, IPO-3), BLAME (SLAMF8), SELPLG (CD162), LTBR, LAT, GADS, SLP-76, PAG/Cbp, CD19a, and a ligand that specifically binds with CD83. Additional co-stimulatory domains will be apparent to those of skill in the art.

The CAR also includes an intracellular signaling domain. This domain may be cytoplasmic and may transduce the effector function signal and direct the cell to perform its specialized function. Examples of intracellular signaling domains include, but are not limited to, chain of the T-cell receptor or any of its homologs (e.g., ζ chain, FcεRIγ and β chains, MB1 (Igα) chain, B29 (Igβ) chain, etc.), CD3 polypeptides (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28. The intracellular signaling domain may be human CD3c chain, FcγRIII, FcεRI, cytoplasmic tails of Fc receptors, immunoreceptor tyrosine-based activation motif (ITAM) bearing cytoplasmic receptors or combinations thereof.

The CAR also includes a transmembrane domain. The transmembrane domain may be the transmembrane sequence from any protein which has a transmembrane domain, including any of the type I, type II, or type III transmembrane proteins. The transmembrane domain of the CAR of the invention may also be an artificial hydrophobic sequence. The transmembrane domains of the CARs of the invention may be selected so as not to dimerize. Examples of transmembrane (TM) regions used in CAR constructs may be obtained from CD28, OX40, 4-1BB, CD3ζ, or CD8a. Additional transmembrane domains will be apparent to those of skill in the art.

An exogenous nucleic acid molecule may be introduced into T cells of the invention by a vector such as an adenovirus, retrovirus, or lentivirus-based vector, or endonucleases, such as CRISPR-associated (CRISPR/Cas9, Cpfl, and the like) nucleases. Other suitable delivery systems include, e.g., DNA transfection methods such as electroporation, DNA biolistics, lipid-mediated transfection, compacted DNA-mediated transfection, liposomes, immunoliposomes, cationic agent-mediated transfection, cationic facial amphiphiles (CFAs) and combinations thereof. Preferably said exogenous nucleic acid molecule is placed at an endogenous gene locus of the T cell. Preferably said introduction of the said exogenous nucleic acid molecule disrupts or abolishes expression of an endogenous TCR.

In an alternative aspect, T cells of the invention are specific for an antigen, e.g., a pathogen or tumor antigen as described herein, by culturing the cells in the presence of an antigen. By way of illustration, tumor antigen-specific CD8⁺ T cells may be generated by culturing lymphocytes from PBMCs in the presence of a tumor antigen, an antigen presenting cell such as a dendritic cell, IL-21, IL-15, and rapamycin and preferably in the absence of IL-2.

The invention also provides population of T cells or engineered T cells produced or obtainable by the methods of the invention. Preferably said produced or obtained T cells or engineered T cells are isolated (i.e., at least 90%, 95%, 97%, 98%, 99% or 99.9% homogenous to said T cells). The invention also provides a population of CAR T cells obtainable by the method of the invention or a population of TCR-engineered T cells obtainable by the method of the invention. Ideally, the population of T cells or engineered T cells of this invention have reduced levels of c-Myc, e.g., at least 20%, 30%, 40%, 50%, 60%, 70%, or 80% reduced levels as compared to the same T cells not contacted with an eIF4A inhibitor, thereby promoting memory-like T cell fate of said T cells.

For therapeutic applications, it is preferable that the population of T cells or engineered T cells described herein are prepared in the form of a pharmaceutical composition including said T cells in admixture with a pharmaceutically acceptable carrier or vehicle. A “pharmaceutical composition” refers to a composition formulated in pharmaceutically acceptable or physiologically acceptable solutions for administration to a cell or an animal, either alone, or in combination with one or more other modalities of therapy. It will also be understood that, if desired, the compositions of the invention may be administered in combination with other agents as well, such as, e.g., cytokines, growth factors, hormones, small molecules, chemotherapeutics, pro-drugs, drugs, antibodies, or other various pharmaceutically active agents. The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Suitable pharmaceutically acceptable carriers or vehicles of use in this invention include without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular aspects, compositions of the present invention include an effective amount of the T cells prepared by the methods described herein. It can generally be stated that a pharmaceutical composition including the T cells prepared by the methods of this invention may be administered at a dosage of 10² to 10¹⁰ cells/kg body weight, preferably 10⁵ to 10⁶ cells/kg body weight, including all integer values within those ranges. The number of cells will depend upon the ultimate use for which the composition is intended as will the type of cells included therein. For uses provided herein, the cells are generally in a volume of a liter or less, can be 500 mL or less, even 250 mL or 100 mL or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. The cells may be allogeneic, syngeneic, xenogeneic, or autologous to the patient undergoing therapy. If desired, the treatment may also include administration of mitogens (e.g., PHA) or lymphokines, cytokines, and/or chemokines (e.g., IFN-γ, IL-2, IL-7, IL-15, IL-12, TNF-α, IL-18, TNF-β, GM-CSF, IL-4, IL-13, Flt3-L, RATES, MIPIα, etc.) to enhance engraftment and function of infused T cells.

Pharmaceutical compositions of the present invention are preferably formulated for parenteral administration, e.g., intravascular (intravenous or intraarterial), intraperitoneal or intramuscular administration. In one aspect, the pharmaceutical compositions are administered intravenously.

In some aspects, pharmaceutical compositions of the invention include an effective amount of an expanded population of T cells, alone or in combination with one or more therapeutic agents. Thus, the T cells may be administered alone or in combination with other known cancer treatments, such as radiation therapy, chemotherapy, transplantation, immunotherapy, hormone therapy, photodynamic therapy, etc. The T cells may also be administered in combination with antibiotics. Such therapeutic agents may be accepted in the art as a standard treatment for a particular disease state as described herein, such as a particular cancer. Exemplary therapeutic agents contemplated include cytokines, growth factors, steroids, NSAIDs, DMARDs, anti-inflammatories, chemotherapeutics, radiotherapeutics, therapeutic antibodies, or other active and ancillary agents.

Generally, compositions including the cells activated and expanded as described herein may be used in a subject (e.g., a mammal such as a human or primate) in need of adoptive T cell therapy. “Adoptive T cell therapy” (or adoptive cell transfer or cellular adoptive immunotherapy or T-cell transfer therapy) refers to a type of immunotherapy in which T cells are given to a subject to help the body fight diseases. Typical subjects include humans that have a cancer, infectious disease, immunodeficiency, inflammatory disease, or auto-immune disorder, which have been diagnosed with a cancer, infectious disease, immunodeficiency, inflammatory disease, or auto-immune disorder, or that are at risk or having a cancer, infectious disease, immunodeficiency, inflammatory disease, or auto-immune disorder. Use of the cells prepared in accordance with the methods described herein increase persistence and better response to the T cells in subjects treated with the same as compared to subjects treated with conventional T cells (i.e., T cells not contacted with an eIF4A inhibitor).

In certain aspects, compositions including the T cells prepared by the methods described herein are used in the treatment of various conditions including, without limitation, cancer, infectious disease, autoimmune disease, inflammatory disease, and immunodeficiency. As used herein, “treatment” or “treating” includes any beneficial or desirable effect on the symptoms or pathology of a disease or pathological condition and may include even minimal reductions in one or more measurable markers of the disease or condition being treated, e.g., cancer. Treatment can involve optionally either amelioration of, or complete reduction of, one or more symptoms of the disease or condition, or the delaying of the progression of the disease or condition. “Treatment” does not necessarily indicate complete eradication or cure of the disease or condition, or associated symptoms thereof.

In particular, the cells of this invention are of use in the treatment of solid tumors or cancers including, without limitation, liver cancer, bone cancer, pancreatic cancer, lung cancer, breast cancer, bladder cancer, brain cancer, bone cancer, thyroid cancer, kidney cancer, ovarian cancer, colon cancer, testicular cancer, head and neck cancer, stomach cancer, cervical cancer, rectal cancer, esophageal cancer, uterine cancer, prostate cancer or skin cancer. Moreover, the cells of the invention are of use in the treatment of leukemia, including acute leukemia (e.g., acute lymphocytic leukemia (ALL), acute myelocytic leukemia (AML), and myeloblasts, promyelocytic, myelomonocytic, monocytic and erythroleukemia), chronic leukemia (e.g., chronic lymphocytic leukemia (CLL), small lymphocytic lymphoma (SLL), chronic myelogenous leukemia (CML), Hairy cell leukemia (HCL)), polycythemia vera, lymphoma, Hodgkin's disease, non-Hodgkin's lymphoma, multiple myeloma, Waldenstrom's macroglobulinemia, and heavy chain disease.

In addition to cancer, a variety of diseases or conditions may be ameliorated by introducing the T cells of the invention to a subject in need of adoptive T cell therapy. Examples of diseases including various autoimmune disorders such as alopecia areata, autoimmune hemolytic anemia, autoimmune hepatitis, dermatomyositis, diabetes (type 1), some forms of juvenile idiopathic arthritis, glomerulonephritis, Graves' disease, Guillain-Barre syndrome, idiopathic thrombocytopenic purpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjogren's syndrome, systemic lupus, erythematosus, some forms of thyroiditis, some forms of uveitis, vitiligo, granulomatosis with poly angiitis (Wegener's); and infections, including but not limited to, HIV (human immunodeficiency virus), RSV (Respiratory Syncytial Virus), EBV (Epstein-Barr virus), CMV (cytomegalovirus), HBV (hepatitis B virus), HCV (hepatitis C virus), adenovirus, coronavirus (e.g., SARS-CoV2) and BK polyomavirus infections.

To facilitate the preparation of T cells for adoptive T cell therapy, the invention also provides a kit including (a) an eIF4A inhibitor as described herein, and (b) a CD3 agonist, as described herein. In some aspects, the kit further includes one or more costimulatory ligands, e.g., an anti-CD28 antibody, and optionally one or more cytokines, e.g., IL-2, IL-15, IL-7, IL-23, or a combination thereof. Kits typically include a label indicating the intended use of the contents of the kit and instructions for use. The term “label” includes any writing, or recorded material supplied on or with the kit, or which otherwise accompanies the kit. In some aspects, the instructions provide the steps used in preparing T cells for adoptive T cell therapy including obtaining a suitable population of T cells (e.g., autologous/autogeneic CD8⁺ T cells isolated from PBMCs of a subject diagnosed with cancer); contacting the T cells with the CD3 agonist (e.g., an anti-CD3 antibody) optionally in the presence of a costimulatory ligand (e.g., an anti-CD28 antibody) for a time sufficient to activate the T cells; and contacting the activated T cells with an effective amount of an eIF4A inhibitor to produce T cells with memory-like potential. The instructions can further include steps for expanding the T cells during and/or after activation with one or more cytokines (e.g., IL-2 or IL-15); introducing exogenous nucleic acid molecules (e.g., a nucleic acid molecule encoding a CAR); and administering the cells to a subject in need of treatment. The kit can also optionally include culture vessels, culture medium, wash solutions, and the like.

The following non-limiting examples are provided to further illustrate the present invention.

### Example 1: Materials and Methods

Mice. BCM mice were as described in the art (Gerlach et al. (2013) Science 340:635-639). GFP-c-Myc fusion knock-in mice have been previously described (Huang et al. (2008) Eur. J. Immunol. 38:342-349). OT-I Tg mice (C57BL/6-Tg (TcraTcrb)1100Mjb/J) were acquired from The Jackson Laboratory (Bar Harbour, ME). All animal experiments were performed with both female and male sex- and age-matched littermate controls (6-10 weeks old). All mice were bred and housed in specific pathogen-free facilities, in a 12-hour light/dark cycle in ventilated cages, with chow and water supply ad libitum, at the Animal Resources Center at St. Jude Children's Research Hospital. Mouse studies were conducted in accordance with protocols approved by the at St. Jude Children's Research Hospital Committee on Care and Use of Animals and in compliance with all relevant ethical guidelines.

Murine CD8⁺ T Lymphocytes. For CD8⁺ T cell stimulation, splenocytes obtained from wild-type animals were enriched for antigen presenting cells (APCs) using CD11c MicroBeads (Miltenyi Biotec) and pulsed with 5 nM of SIINFEKL peptide (SEQ ID NO:1; AnaSpec Inc.) for 1 hour at 37° C. CD8⁺ T cells were isolated from lymph nodes using the Dynabeads Untouched Mouse CD8 Cells Kit (Invitrogen) and overlain onto the peptide-pulsed APCs. Alternatively, CD8⁺ T cells were stimulated on plate-bound anti-CD3ε (1 μg/ml, Bio X Cell), anti-CD28 (1 μg/ml, Bio X Cell), and recombinant human ICAM (0.5 μg/ml) produced in insect cells (Huppa et al. (2010) Nature 463:963-967); or by incubation with PMA (10 ng/ml, MedChemExpress) and Ionomycin (1 μM, MedChemExpress). Whenever assessment of cell division was required, CD8⁺ T cells were labeled with a fluorescent dye sold under the tradename CELLTRACE® Violet (CTV) Cell Proliferation Kit (Invitrogen) at 5 μM per 1×10⁷ cells for 10 minutes at 37° C. For in vivo stimulation, CTV-labeled CD8⁺ T cells were transferred by tail vein injection of wild-type recipients. Twenty-four hours after transfer, recipients were immunized by subcutaneous injection of 50 μg of SIINFEKL peptide (SEQ ID NO:1) into the neck region. Donor cells were obtained from axillary and brachial lymph nodes 8 hours after immunization. CD8⁺ T cells were cultured in RPMI 1640 (Gibco) supplemented with 10% (v/v) heat-inactivated fetal bovine serum (FBS), lx MEM non-essential amino acids (Gibco), 1 mM Sodium pyruvate (Gibco), 2 mM L-glutamine (Gibco), 55 μM 2-mercaptoethanol (Gibco), 50 U/ml penicillin and 50 μg/ml streptomycin (Corning) at 37° C. in 5% CO₂. For all imaging, CD8⁺ T cells were cultured in 4-well p-slides (ibidi). Where indicated, CD8⁺ T cells were treated with silvestrol (200 nM, MedChemExpress), Hippuristanol (500 nM), Torin 1 (1 μM, Selleckchem), Dynarrestin (25 μM, Sigma), or Ciliobrevin D (50 μM, MedChemExpress) for the last hour of activation, or Puromycin (91 μM, Tocris).

Single-Cell RNA Sequencing (scRNA-Seq). OT-I BCM CD8⁺ T cells labeled with CTV were stimulated on peptide-pulsed APCs for 36 hours. Cells were stained with anti-CD8-APC (Invitrogen) and anti-CD44-PE/Cy7 (BioLegend) for sorting on a MoFlow (Beckman-Coulter). For in vivo stimulation, CTV-labeled CD8⁺ T cells were transferred by tail vein injection of wild-type recipients. Twenty-four hours after transfer, recipients were immunized by subcutaneous injection of 50 μg of SIINFEKL peptide (SEQ ID NO:1) into the neck region. Donor cells were obtained from spleens and peripheral lymph nodes 24 hours after immunization. First-division CD8⁺ T cells expressing a barcode (CD8⁺, CD44⁺, CTV 2^{nd} peak, GFP-bcm⁺) were sorted into culture medium, washed once with PBS+0.04% BSA, and re-suspended in 32 μl PBS+0.04% BSA. Single-cell suspensions were loaded onto the Chromium Controller to generate up to 10000 single-cell gel beads in emulsion (GEMs) per sample. Single-cell gene expression libraries were prepared using the Chromium Single Cell 5′ v2 Library and Gel Bead Kit (10× Genomics) for BCM experiments and the 3′ v2 kit for non-BCM experiments. Resulting libraries were sequenced on Illumina HISEQ® or NOVASEQ® platforms at 26×98 bp.

Barcode-containing transcripts were enriched from amplified cDNA via target enrichment PCR amplification (1^{st} fwd—AAT GAT ACG GCG ACC GAG ATC TAC ACT CTT TCC CTA CAC GAC GCT C (SEQ ID NO:2); 1^{st} rev—GCT GAA CTT GTG GCC GTT TA (SEQ ID NO:3); 2^{nd} fwd—AAT GAT ACG GCG ACC GAG ATC T (SEQ ID NO:4); and 2^{nd} rev—CGT CCA GCT CGA CCA GGA T (SEQ ID NO:5)) to create a separate single-cell barcode library (Kapa HyperPrep Kit), which was sequenced at 250×250 bp. Quality controls and quantifications were performed using High Sensitivity D5000 Screen Tape (Agilent Technologies) with a 4200 TapeStation (Agilent Technologies).

Analysis of scRNA-Seq Data. The single-cell data can be divided into two broad experiments, each with independent replicates. For the barcode experiments, single-cell gene expression data were processed with CellRanger (v3.1.0, 10× Genomics) using the accompanying mouse transcriptomic reference (v3.0.0), and the processed libraries were aggregated after normalizing for the number of confidently mapped reads per cell (BCM and non-BCM libraries were aggregated separately due to the distinct molecular chemistry used to generate each). Filtered aggregation outputs were subsequently analyzed using Seurat (v4.0.2) (Hao et al. (2021) Cell 184:3573-3587) and following standard procedures. For BCM libraries, the aggregated data were used to create a Seurat object with 10,149 putative cells (3,511 and 6,638 for each replicate, respectively) and 12,038 genes (only retaining genes found in a minimum of 0.3% of cells, i.e., ˜30 cells). Independent replicates exhibited similar distributions of typical QC features, including number of genes per cell, number of RNA molecules per cells, and percent of mitochondrial expression per cell; however, the replicate with the larger number of cells exhibited slightly more cells that fell outside the general range of genes per cell and RNA molecules per cell, as expected given the increasing frequency of cell multiplets as more cells are recovered from a 10×reaction. Filtering thresholds were decided based on the distributions of these QC metrics. To exclude putative multiplets, cells with more than 6,000 genes or more than 42,000 Unique Molecular Identifiers (UMIs) were filtered out. Cells with >7.5% of expression owed to mitochondrial genes were also filtered out to exclude dead or dying cells. A population of cells was subsequently identified with relatively few genes and very low mitochondrial expression (a feature commonly observed in some versions of the CellRanger cell calling algorithm). To exclude these transcriptionally inactive cells and false cell calls, cells with <0.1% of expression owed to mitochondrial genes and cells with fewer than 300 genes were filtered out. After filtering, 2,192 and 3,771 cells per replicate, respectively, remained; importantly, these filtered data exhibited expected relationships between the observed number of genes and mitochondrial expression and between the number of UMIs and number of genes.

Data were then LogNormalized within Seurat using a scale.factor of 1e4, and cell cycle phase was inferred using the CellCycleScoring function with known markers (Tirosh et al. (2016) Science 352:189-196). After filtering the two independent replicates together to ensure no biases in these processing steps, the two independent replicates were then split from the filtered, normalized Seurat object to be analyzed independently. This was done to demonstrate independent replication of the patterns that were observed. For each independent replicate, the data were separately scaled, the number of UMIs and the percent of expression owed to mitochondrial genes in each cell were regressing out. The ‘vst’ method was then used to find 2,000 variable features (again, separately for each replicate), after excluding any possible variable features owing to V(D)J gene segments, which are known to map poorly. Those variable features were used for Principal Component Analysis (PCA) and UMAP analysis (with min.dist=0.5) for each replicate.

To broadly survey variation in major functional pathways, expression modules were utilized from a number of curated gene sets, including the entire Hallmark collection (obtained from the msigdbr package in R; (Subramanian et al. (2005) Proc. Natl. Acad. Sci. USA 102:15545-15550), two gene sets previously described in the context of asymmetrical T cell division (Kakaradov et al. (2017) Nat. Immunol. 18:422-432), and a gene set comprised of c-Myc target genes involved in metabolic processes (Wang et al. (2011) Immunity 35:871-882). To do this, the AddModuleScore function in Seurat was used for each gene set within each independent Seurat object. To match the maximum number of genes between the gene sets and the mouse reference, the UpdateSymbolList function was directly used rather than using the search=TRUE parameter in the AddModuleScore function.

Once each cell was assigned a module score for each gene set, differences between cell-cell pairs were identified. In the barcode experiments, for putative sister cells specifically, absolute differences in each module between the two sister cells were calculated. Distances between cell pairs were also calculated using Euclidean distances between PC1 and PC2 (using the pointDistance function in the raster R package). Correlations were assessed using Spearman correlations (specifically using the cor.test function with method=“Spearman” and use=“compleWe.obs”), and p-values were adjusted to a False Discovery Rate (FDR)<0.05.

Barcode-containing transcripts were enriched from amplified cDNA via target enrichment PCR to create single-cell barcode libraries, which were sequenced at 250×250 bp and processed with CellRanger VDJ (v3.0.2) using a custom reference based on the potential barcode segments and arrangements present in the endogenous BCM system (Gerlach et al. (2013) Science 340:635-639). Filtered contigs from the VDJ assemblies were aligned to the genomic sequence corresponding to the region of barcode rearrangement using MUSCLE (v3.8.31) (Edgar (2004) Nucl. Acids Res. 32:1792-1797). The resulting alignments were parsed with custom python scripts to trim the contigs to only span between the conserved regions immediately upstream (TTACCTCCTCGAGGTCA; SEQ ID NO:6) and downstream (CATGGTGAGCAAGGGC; SEQ ID NO:7) of the rearrangements.

Because of the known limitations of this BCM system, patterns of convergent recombination among cells within each experiment were characterized by identifying barcodes that appeared more than once in an undivided population, more than twice in a first-division population, or three or more times in undivided and first-division populations from the same experiment. To this end, BCM OT-I transgenic CD8⁺ T cells were activated on APCs pulsed with the OT-I T cell cognate antigen SIINFEKL (SEQ ID NO:1) for 36 hours. CD8⁺ T cells expressing a barcode (CD8⁺, CD44⁺, GFP-bcm⁺) were sorted by flow cytometry into undivided, first and second division cell populations based on CTV intensity followed by preparation and sequencing of single-cell barcode libraries using 10× genomics technology. In total, 7,975 cell-specific barcodes across 11 barcode libraries derived from either undivided, first-division, or second division cells were sequenced. Overall, barcodes were assembled from a median of 51,891 sequencing reads derived from a median of 56 unique transcripts (range: 1-557 UMIs) per cell. Convergent rearrangements were responsible for approximately half of the barcodes sequenced, but the majority of these problematic barcodes were derived from a limited number of rearrangements; for instance, more than 43% of the problematic barcodes identified were derived from a single common rearrangement that was only observed in a single experiment. The 62 barcodes that were each shared between a single pair of first-division cells were assembled from a median of 52,116 reads (range: 6,321-178,130) derived from a median of 39.5 unique transcripts (range: 8198 UMIs) per cell and exhibited variation in length ranging from 92 to 132 bp.

To address if putative sisters that were transcriptionally different may have arisen from distinct lineages and were only sharing a barcode as a result of instances of convergent barcode rearrangement that could not be excluded, a probabilistic model of V(D)J rearrangement specifically for this BCM system was generated. Probabilities of generation (P_{gen}) for barcode sequences were estimated with IGoR (Marcou et al. (2018) Nat. Commun. 9:561), using the BCM segments and potential arrangements as a custom mouse TCR reference. The recombination model was inferred using the total set of barcode sequences (including duplicates), with a probability ratio threshold of 1e-20, a sequence likelihood threshold of 1e-100, and 10 expectation-maximization algorithm iterations. Using this model, finite estimates of P_{gen} for >99% of unique barcode sequences were generated.

Whereas barcodes identified as problematic exhibited a median probability of generation of approximately 1.8 in 100,000, all other barcodes had much lower P_{gen} estimates, with a median probability of approximately 6 in 100 million. In contrast, a P_{gen} of 7 in 10,000 was estimated for the most frequently observed convergent rearrangement across experiments.

Single-cell data from first-division cells activated in vivo were generated using 3′ v2 10×kits. The data were processed in the same manner as described above. Filtering thresholds were changed slightly to reflect the distinct distributions of the QC metrics for each library.

Immunofluorescence Staining and Imaging. CD8⁺ T cells were stimulated on peptide-pulsed APCs for 28 hours followed by fixation for 10 minutes at room temperature (RT) with paraformaldehyde (PFA) (Electron Microscopy Science) directly added to the culture medium at 4% (v/v) final concentration. Cells were rinsed with TBS (50 mM Tris pH 8 (Sigma-Aldrich), 100 mM Sodium Chloride (Sigma-Aldrich) in ddH₂O) and permeabilized with TBS+0.1% (v/v) TRITON X-100 (Sigma-Aldrich) for 3 minutes at RT. Non-specific binding was blocked with TBS+2% BSA (Sigma-Aldrich) for 30 minutes at RT. Samples were incubated overnight at 4° C. with the following primary antibodies: anti-c-Myc (1:500, Cell Signaling Technology), anti-Sirt1 (1:500, Abcam), anti-eIF4A1 (1:500, Abcam), anti-phospho-eIF4GI^{S1108} (1:500, Cell Signaling Technology), anti-eIF4GI (1:500, Cell Signaling Technology), anti-phospho-mTOR^{S2481} (1:500, Cell Signaling Technology), anti-mTOR (1:500, Cell Signaling Technology), anti-CD11c (1:500, BioLegend), anti-tubulin (1:1000, Invitrogen), anti-S6 (1:500, Cell Signaling Technology), anti-tubulin (1:1000, Invitrogen), and anti-tubulin (1:1000, Invitrogen). Samples were washed with TBS and incubated for 1 hour at RT with the following secondary antibodies: Donkey anti-mouse AFplus488 (1:1000, Invitrogen), donkey anti-rabbit AFplus555 (1:1000, Invitrogen), donkey anti-rat AF647plus (1:1000, Jackson ImmunoResearch), and goat anti-hamster AF488 (1:500, Jackson ImmunoResearch).

For imaging of in vivo stimulated CD8⁺ T cells, axillary and brachial lymph nodes were fixed in PBS containing 2% PFA, 0.1% TRITON X-100, and 1% DMSO overnight at 4° C. Tissues were subsequently cryosectioned onto charged glass slides. Sections were blocked in PBS containing 1% BSA prior to incubation with primary antibodies mentioned above at 1:250 dilution overnight at 4° C. Slides were washed in PBS and incubated for 1 hour at RT with secondary antibodies mentioned above at 1:500 dilution. Slides were mounted with Prolong Glass Antifade Mountant (Invitrogen).

Detection of c-myc mRNA was facilitated using reagents from Advanced Cell Diagnostics, per the manufacturer's protocol. In brief, antibody-activated cells were fixed with 4% PFA in PBS for 15 minutes, washed, permeabilized with 0.1% TRITON-100 for 3 minutes, and blocked with 1% BSA in PBS for 10 minutes at RT. Cells were subsequently stained with rat anti-tubulin antibody for 15 minutes at RT prior to detection with AF647-conjugated donkey anti-rat secondary (Jackson ImmunoResearch) for 15 minutes at RT. Samples were post-fixed in 1% PFA in PBS prior to dehydration in an ethanol series. Cells were rehydrated to PBS and protease digested for 15 minutes, followed by hybridization with a c-Myc RNA probe (ACD) and detection utilizing a fluorescent reagent (ACD) per the manufacturer's instructions.

For imaging, a Marianas confocal (Intelligent Imaging Innovations) composed of a CSU-X spinning disk, Prime95B sCMOS camera, and 405, 488, 561 and 640 nm laser lines was used. Alternatively, a CSU-W (Yokogawa) spinning disk to facilitate super resolution via optical reassignment (SoRA) imaging in combination with a 100×1.45 NA oil objective and Prime 95B camera was used.

Diving sister cells were identified by the presence of an intercellular cytokinetic bridge based on tubulin staining. Using Slidebook 6 imaging software (Intelligent Imaging Innovations), regions for each sister cell were created and converted to masks. Masks statistics of the sum fluorescent intensities were analyzed.

The distribution of proteins in undivided cells was analyzed using Imaris 9.5 software (Oxford Instruments). A surface object and spots were created to segment the microtubule-organizing center (MTOC) and the indicated proteins, respectively. The distance of protein to the MTOC was measured via intensity to distance transformation and the proportion of protein in the proximal vs. distal half of the cell was calculated. Three independent experiments with at least 10 undivided cells or sister cell pairs each were analyzed.

Stochastic Optical Reconstruction Microscopy (STORM). For STORM analysis of translating polysomes in vitro, CD8⁺ T cells were stimulated on peptide-pulsed APCs in a 96-well round bottom plate. PFA was added to the culture at a final concentration of 4% (v/v), the cell suspension was transferred into 1.5 μl tubes, and fixation continued for 10 minutes at RT. During all sample preparation, cells were pelleted for 3 minutes at 2000 rpm between each buffer. Cells were permeabilized in TBS+0.1% (v/v) TRITON X-100 for 3 minutes at RT. Reactive groups were quenched in 20 mM glycine, and non-specific antibody binding was blocked in TBS+2% BSA. Samples were incubated overnight at 4° C. with the following antibodies: anti-c-Myc (1:500, Cell Signaling Technology), anti-RPL26 (1:1000, Sigma-Aldrich), anti-Sirt1 (1:500, Abcam), anti-tubulin (1:1000, Invitrogen). Cells were washed with TBS and incubated with the following secondary antibodies for 1 hour at RT: CF488-labeled donkey anti-rat (1:1000, Biotium), CF568-labeled donkey anti-Goat (1:1000, Biotium), and AF647-labeled donkey anti-rabbit (1:1000, Jackson ImmunoResearch). Following two washes with TBS, cells were post-fixed in 1% (v/v) PFA for 5 minutes at RT, pelleted and resuspended in 20 μl cell culture matrix sold under tradename GELTREX® (Gibco). A 10 μl drop of the cell suspension was placed into a cryomold, overlaid with Tissue Freezing Medium (Fisher Scientific Company) and snap frozen on dry ice. Ten μl thick cryosections were placed on poly-L-lysine coated #1.5 18-mm coverslips (Electron Microscopy Sciences) and affixed to 35-mm dishes (Mattek) using epoxy resin. Stochastic optical reconstruction microscopy was performed using an N-STORM system (Nikon Instruments) as previously reported (Liedmann et al. (2014) Nat. Commun. 5:5645). Co-ordinate (x,y) positions of identified single molecules were exported for analysis. At least 25 cells from two independent experiments were analyzed.

For STORM analysis of translating polysomes in vivo, 24 h after immunization axillary and brachial lymph nodes were harvested and fixed in PBS containing 2% PFA, 0.1% Triton X-100 and 1% DMSO overnight at 4° C. 10 um thick cryosections were placed on poly-L-lysine coated coverslips and affixed to MatTek dishes as described above. Sections were blocked in PBS containing 1% BSA prior to incubation with primary and secondary antibodies as described above. 6 cells from two independent experiments were analyzed.

Analysis for STORM Data. After the removal of noise signals from outside the cell borders and the nucleus the Density-Based Spatial Clustering of Applications with Noise (DBSCAN) algorithm was used to identify RPL26 and S6 clusters, defined by a proximity size of 100 nm and minimum number of signals of 10 within 100 nm. RPL26 and S6 clusters with c-Myc or Sirt1 molecules in close vicinity of 100 nm were defined as c-Myc or Sirt1 translating polysomes. Next, the physical distances of c-Myc or Sirt1 molecules within RPL26 and S6 clusters from the MTOC were calculated and binned between 0 and the largest distance from the MTOC, where the largest distance from the MTOC is normalized to be 1. The average distances were regressed against counts of c-Myc/Sirt1 molecules in binned groups using Poisson regression with mixed effects across all images analyzed. The estimated slopes and the standard errors in the regressions for the effects of distances from the MTOC onto the counts of c-Myc/Sirt1 molecules were used to test the polarization of c-myc or sirt1 translating ribosomes towards the MTOC.

Rigorous statistical analysis of single molecule data, allowing statistical comparison across groups, required the development of a new algorithm denoted ‘normalized spatial intensity correlation’ (NSInC). Briefly, co-ordinate data of identified single molecules were analyzed for the degree of bi-directional co-localization using the NSInC algorithm, which incorporates terms for normalization and correction of edge effect, and which is determined to be unbiased under conditions of complete spatial randomness. According to the algorithm, a value of 0 corresponds to complete spatial randomness between the molecules in question, while a value of 1 corresponds to complete co-localization and a value of −1 represents complete exclusion of molecules within the two-dimensional or three-dimensional study region. The NSInC algorithm was applied to STORM data sets containing coordinate data for vATPase and p-eIF4GI wherein multiple regions of interest (ROI) were analyzed per cell, with ROI defined by the inclusion of at least 3 vATPase⁺ cytoplasmic lysosomal structures.

Simulated Emission Depletion (STED) Microscopy. For super resolved confocal imaging, cells were imaged with a 100×1.4NA oil objective on a Leica TCS SP8 STED 3× microscope equipped with a 405 nm diode laser, a tunable (470-670 nm tunability range) white light laser and three STED depletion lasers with wavelength of 592 nm, 660 nm and 775 nm. Hoechst channel was excited with 405 nm diode laser and detected with a PMT detector in the 415-478 nm range. Fluorescent dyes sold under the tradenames ALEXA FLUOR® 488 (ALEXA-488), ALEXA FLUOR® 555 (ALEXA-555), and ALEXA FLUOR® 594 (ALEXA-594), and Atto-647N dye-labeled probe channels were excited at 494 nm, 552 nm, 590 nm, and 650 nm wavelengths, respectively, using the white light laser and detected with Leica GaAsP HyD detectors in 498-536 nm, 556-584 nm, 595-645 nm, and 655-740 nm ranges, respectively. ALEXA-594 and Atto-647N dye-labeled probe channels were imaged in 3-D STED mode using 775 nm depletion laser with pinhole diameter set to 0.8 Airy unit calculated for the lowest wavelength of the two dyes. ALEXA-488 and ALEXA-555 dye-labeled probe channels were imaged in confocal mode with pinhole diameter set to 0.4 Airy unit calculated for the lowest wavelength of the two dyes. Hoechst channel was imaged in confocal mode with pinhole diameter set to 0.8 Airy unit. All images were acquired with LAS X software (version 3.5) as 3-D stacks with 22 nm XY pixel size and either 120 or 150 nm Z-step size. Post-acquisition, the images were processed with built-in Lightning adaptive deconvolution module with optimized settings for each channel.

Expansion Microscopy. Expansion microscopy was performed as recently described (Zhang et al. (2020) Curr. Protoc. Neurosci. 92:e96). Briefly, cells were cultured for 2 hours on anti-CD3/CD28-coated 12-mm glass coverslips, prior to fixation with 4% PFA in PBS for 15 minutes. Cells were subsequently permeabilized with 0.1% TRITON X-100 for 3 minutes and blocked with 1% BSA in PBS for 30 minutes prior to incubation with primary antibodies overnight at 4° C. Samples were washed in PBS prior to detection with the following secondary antibodies: anti-mouse 488plus (ThermoFisher), anti-rabbit 488plus (ThermoFisher), CF568 conjugated anti-rat (Biotium), or Atto647N conjugated anti-rat (Rockland). Following incubation with secondary antibodies, samples were washed in PBS prior to incubation with 0.1 mg/ml acryloyl-X-SE (ThermoFisher) overnight at 4° C. Samples were subsequently washed in PBS prior to gelation with an acrylate/acrylamide solution, followed by proteinase K digestion as described previously (Zhang et al. (2020) Curr. Protoc. Neurosci. 92:e96). Samples were finally washed three times in water to facilitate expansion and were visualized using SoRa.

Adoptive Transfer and in vivo Infection. To study the functional consequence of brief eIF4A inhibition in vivo, CTV-labeled GFP-c-Myc, OT-I⁺ CD45.2⁺ CD8⁺ T cells were activated on peptide-pulsed APCs for 36 hours. First-division GFP-c-Myc^{high} and GFP-c-Myc^{low} cell populations (CD8⁺, CTV 2nd peak, highest and lowest 15% GFP-c-Myc) were sorted into medium with or without 200 nM silvestrol and cultured for additional 2 hours. Next, 5×10⁵ cells were transferred i.v. into CD45.1⁺ recipient animals, which were infected with 4000 EID₅₀ influenza A virus A/X-31 (H3N2) expressing SIINFEKL peptide (SEQ ID NO:1). Thirty days later animals were challenged with 10⁵ EID₅₀ influenza A virus A/PR/8 (H1N1) expressing SIINFEKL peptide (Zhang et al. (2020) Curr. Protoc. Neurosci. 92:e96). Nine days after the secondary infection spleens were collected and analyzed by flow cytometry for the presence of donor cells.

Flow Cytometry. Cells were stained for 20 minutes at 4° C. in PBS+5% BSA with the following antibodies from eBioscience at 1:300 dilutions: Anti-CD8-PerCP5.5, anti-CD45.1-PE, anti-CD45.2-FITC, anti-TCR-Va2-APC, anti-CD44-PE-Cy7, and anti-CD62L-BV605 (BioLegend). Samples were acquired on a SP6800 Spectral Cell Analyzer (Sony Biotechnology) and analyzed using FlowJo 10.1r7 software (FlowJo, LLC).

CHIP-Sequencing. First-division CD8⁺ T cells were sorted into medium with or without 200 nM silvestrol and cultured for additional 2 hours. CUT&RUN ChIP-seq experiments were performed as previously described (Meers et al. (2019) Elife 8) with slight modifications. Briefly, 3×10⁵ cells were washed with Wash Buffer (20 mM HEPES (Sigma-Aldrich), 150 mM NaCl (Invitrogen), 0.5 mM spermidine (Sigma-Aldrich), and Protease inhibitor cocktail (Sigma-Aldrich) twice. Cells were then resuspended and bound to Concanavalin A-coated magnetic beads (Bang Laboratories). Samples were placed on the magnet stand to pull off the liquid and beads were resuspended in 100 μl antibody buffer (20 mM HEPES, 150 mM NaCl, 0.5 mM spermidine, 0.01% digitonin (Millipore), 2 mM EDTA (Invitrogen), and Protease inhibitor cocktail). Anti-c-Myc antibody (Cell Signaling Technology) was added to the samples at a final concentration of 1:100 and incubated overnight at 4° C. The next day, samples were washed with cold Dig-wash buffer (20 mM HEPES, 150 mM NaCl, 0.5 mM spermidine, 0.01% digitonin and protease inhibitor) twice. After washing, pAG-MNase (Addgene) was added to tubes and rotated at 4° C. for 1 hour. After two washes with Dig-wash buffer, samples were resuspended in 50 μl Dig-Zash buffer, 2 μl of 100 mM CaCl₂ (Sigma-Aldrich) was added per sample, briefly vortexed, and immediately placed on ice for 30 minutes. Fifty μl 2χSTOP buffer (340 mM NaCl, 20 mM EDTA, 4 mM EGTA (Sigma-Aldrich), 100 μg/mL RNAse A (Thermo-Fisher), and 50 μg/mL GlycoBlue (Invitrogen)) were added and mixed by gentle vertexing. Then, samples were incubated 30 minutes at 37° C. to release CUT&RUN fragments. Fragmented DNA was purified with the NEB Monarch PCR&DNA Cleanup Kit (NEB). DNA libraries were prepared using the NEBNext Ultra II DNA Library Prep Kit (NEB) and purified with AMPure SPRI beads (Beckman-Coulter). Libraries were quantified using Qubit and size distribution was determined by Agilent 4200 TapeStation analysis before 15 million paired-end sequencing was performed.

Analysis for CHIP-seq Data. Paired-end sequencing reads were trimmed with Trim Galore (version 0.5.0) with default parameters. Reads were aligned to the reference mouse mm10 assembly using Bowtie 2 (version 2.3.5.1) (Langmead & Salzberg (2012) Nat. Methods 9:357-359) with settings—end-to-end—very-sensitive—nomixed—no-discordant-q—phred33-I 10-X 700. The resulting alignments, recorded in BAM file, were sorted, indexed, and marked for duplicates with Picard MarkDuplicate function (version 2.19.0, Broad Institute). The BAM file was filtered with SAMtools (version 1.9) (Li et al. (2009) Bioinformatics 25:2078-2079), BamTools (version 2.5.1) (Barnett et al. (2011) Bioinformatics 27:1691-1692), and scripts of nf-core/chipseq (Ewels et al. (2020) Nat. Biotechnol. 38:276-278) to discard reads, mates that were unmapped, PCR/optical duplicates, not primary alignments, mapped to multiple locations, mapped to ENCODE blacklisted regions (Amemiya et al. (2019) Sci. Rep. 9:9354) or that have more than 4 mismatches (-F 0x004-F 0x008-F 0x0100-f 0x001-q 1). MACS (version 2.1.2) (Zhang et al. (2008) Genome Biol. 9:R137) was used to call peaks from the BAM file with narrowPeak setting, IgG control, and recommended mappable genome size (default value for other parameters). C-Myc occupancy was normalized by scaling to 1 million mapped reads using BEDTools (version 2.27.1) (Quinlan & Hall (2010) Bioinformatics 26:841-842) and bedGraphToBigWig (version 377) (Kent et al. (2010) Bioinformatics 26:2204-2207) and visualized as heatmaps using deepTools plotHeatmap (version 3.2.1) (Ramirez et al. (2016) Nucl. Acids Res. 44:W160-165).

Immunoprecipitation. CD8⁺ T cells were stimulated on peptide-pulsed APCs for 24 hours, treated with Torin for 1 hour, and harvested in cold PBS. Immunoprecipitations were performed using the Dynabeads Co-Immunoprecipitation Kit (Invitrogen) following manufacturer's instructions. In brief, pelleted cells were lysed in lysis buffer supplemented with phosphatases (Roche) and proteases inhibitors (Roche). Primary antibodies against anti-RagC (Cell Signaling Technology) and anti-eIF4GI (Cell Signaling Technology) were covalently coupled to Dynabeads M-270 Epoxy beads. For detection of co-precipitated proteins by western blot, membranes were blocked in 5% BSA, and probed against anti-eIF4A (1:1000, Abcam), anti-Raptor (1:1000, Cell Signaling Technology) or anti-phospho-eIF4G (1:1000, Cell Signaling Technology). Anti-Lamin B1 (1:1000, Abcam) served as loading control. For quantification, densitometric signals of co-immunoprecipitated proteins were normalized with respective signals in the input samples.

RNA Isolation, Reverse Transcription, and qPCR. Total RNA from immunoprecipitated samples spiked with Drosophila mRNA was isolated using RNeasy Mini Kit (Qiagen). First-strand synthesis was performed using M-MLV Reverse Transcriptase (Thermo Fisher Scientific) and oligo(dT). cDNA was preamplified using PreAmp Master Mix (Fluidigm) following manufacturer's instructions. Quantitative amplification was performed using SYBR Green PCR Master Mix in an 7900HT thermocycler (Applied Biosystems). mRNA levels were detected using the following primer sequences: c-myc fwd—TTT GTC TAT TTG GGG ACA GTG TT (SEQ ID NO:8); c-myc rev—CAT CGT CGT GGC TGT CTG (SEQ ID NO:9); dro-rp132 fwd—ATG CTA AGC TGT CGC ACA AAT G (SEQ ID NO:10); dro-rp132 rev—GTT CGA TCC GTA ACC GAT GT (SEQ ID NO:11). mRNA levels were normalized to Drosophila rp132 mRNA level.

Quantification and Statistical Analysis. No statistical tests were used to estimate sample size. Data were plotted and analyzed with GraphPad Prism 8.0 software (GraphPad Software). Statistical significance was calculated with unpaired or paired two-tailed Student's t-test or ANOVA, as specified in the figure legends. Poisson regression was used on imaging data to assess co-localization between two proteins.

Differences were considered statistically significant when the p-value was less than 0.05. For detailed information on statistical analysis of scRNAseq or STORM data please refer to the respective method section.

### Example 2: c-Myc is Synthesized at the Proximal Pole of Activated, Undivided CD8⁺ T Cells

Once translated, c-Myc is imported into the nucleus, suggesting that cytosolic c-Myc signal is the nascent c-Myc peptide or recently synthesized c-Myc protein. To assess the spatial distribution of c-Myc synthesis in undivided T cells, cytosolic c-Myc was visualized using SoRa (Super-Resolution via Optical Reassignment). The distance of cytosolic c-Myc signal to the proximal pole of the cell was then measured using the MTOC as the point of reference. It was found that early during T cell activation, cytosolic c-Myc polarized toward the proximal pole of the cell. In contrast, a similar analysis of cytosolic Sirt1, another protein that translocates to the nucleus, showed no such polarization with respect to the MTOC. As expected, polysomes, assessed by staining of RPL26 (Viero et al. (2015) J. Cell Biol. 208:581-596), were equally distributed throughout the cell. The absence of a significant positive correlation between the distribution of cytosolic c-Myc and RPL26 argues against a significant contribution of uneven cytosolic volumes to the observed proximal polarization of cytosolic c-Myc.

To further assess whether synthesis of c-Myc is localized towards the MTOC, Stochastic Optical Reconstruction Microscopy (STORM) was performed. Using Density-Based Spatial Clustering of Applications with Noise (DBSCAN), polysomes were identified using two antibodies for the small (RPS6) and the large (RPL26) ribosomal subunits and visually confirmed to be the appropriate size and shape (Viero et al. (2015) J. Cell Biol. 208:581-596). Simulated Emission Depletion (STED) microscopy of RPL26 and S6 confirmed a high degree of colocalization, suggesting the assembly of 80S ribosomes, mirroring STORM data in size and shape. Next, it was found that those polysomes associated with c-Myc N-termini were polarized toward the proximal pole of cells activated in vivo or in vitro, as represented by a negative relation between c-Myc and RPL26 co-association and distance to the MTOC. Brief treatment with the protein synthesis inhibitor puromycin significantly reduced the association of c-Myc with RPL26 and S6, providing further evidence that the identified structures are indeed enriched for functional polysomes engaged in active c-myc translation. In contrast, and in agreement with our SoRa results, Sirt1-associated polysomes and polysomes in general were equally distributed throughout the cytosol. Importantly, c-myc mRNA was equally distributed throughout the cell, supporting the idea that synthesis of c-Myc, rather than location of c-myc mRNA, determines the localization of cytosolic c-Myc.

Together, these results indicate that c-Myc synthesis in activated T cells is preferentially localized to the region of the MTOC and argue against a general polarization of cellular content or c-myc mRNA polarization as an explanation for this localized synthesis of c-Myc.

### Example 3: Inhibition of eIF4A Before the First Division Equilibrates c-Myc Level in Sister Cells

The c-Myc protein is generated either by 5′ cap-dependent or IRES-dependent translation (Stoneley et al. (2000) Nucl. Acids Res. 28:687-694). At the initiation of 5′ cap-dependent c-myc translation, the recruitment of ribosomes to mRNAs is mediated by the eukaryotic translation initiation factor 4F (eIF4F), a protein complex composed of three subunits: (i) the cap-binding protein eIF4E; (ii) the RNA helicase eIF4A, needed to unwind local RNA structures to allow for ribosomal scanning of mRNA templates that possess long and highly structured 5′ UTRs (Rubio et al. (2014) Genome Biol. 15:476; Svitkin et al. (2001) RNA 7:382-394; Wolfe et al. (2014) Nature 513:65-70); and (iii) the scaffold protein eIF4G, which binds eIF4E and eIF4A, and recruits the 43S pre-initiation complex to the mRNA (see Pelletier & Sonenberg (2019) Annu. Rev. Biochem. 88:307-335).

The mRNA of c-Myc is characterized by a long and highly structured 5′ UTR, rendering its translation dependent on the activity of eIF4A (Wolfe et al. (2014) Nature 513:65-70). Brief treatment of activated CD8⁺ T cells with silvestrol, a potent inhibitor of eIF4A (Bordeleau et al. (2008) J. Clin. Invest. 118:2651-2660; Cencic et al. (2009) PLoS ONE 4:e5223), greatly reduced c-Myc bound to chromatin at c-Myc binding sites based on chromatin immunoprecipitation, as expected (Rubio et al. (2014) Genome Biol. 15:476; Wolfe et al. (2014) Nature 513:65-70). To test if the asymmetry of c-Myc in first-division sister cells is regulated by eIF4A activity, c-Myc protein distribution was analyzed in cells treated with silvestrol. Strikingly, inhibition of eIF4A immediately prior to the first division resulted in the rapid equilibration of c-Myc levels in first-division sister cells. Similar effects on c-Myc asymmetry were also observed using Hippuristanol, another eIF4A inhibitor (Bordeleau et al. (2008) J. Clin. Invest. 118:2651-2660). In contrast to c-myc, sirt1 mRNA is characterized by a short, unstructured 5′ UTR, the translation of which relies less on eIF4A (Elfakess et al. (2011) Nucl. Acids Res. 39:7598-7609; Sinvani et al. (2015) Cell Metab. 21:479-492). It was determined that Sirt1 protein was equally distributed between dividing sister cells and unaffected by either silvestrol or Hippuristanol treatment. The protein distribution of eIF4A showed no polarization prior to, or asymmetry following first division in activated CD8⁺ T cells. Together, these results demonstrate the importance of eIF4A function for the asymmetric distribution of c-Myc in first division CD8⁺ daughter cells.

### Example 4: Inhibition of eIF4A at the Time of First Division Promotes Memory-Like Function

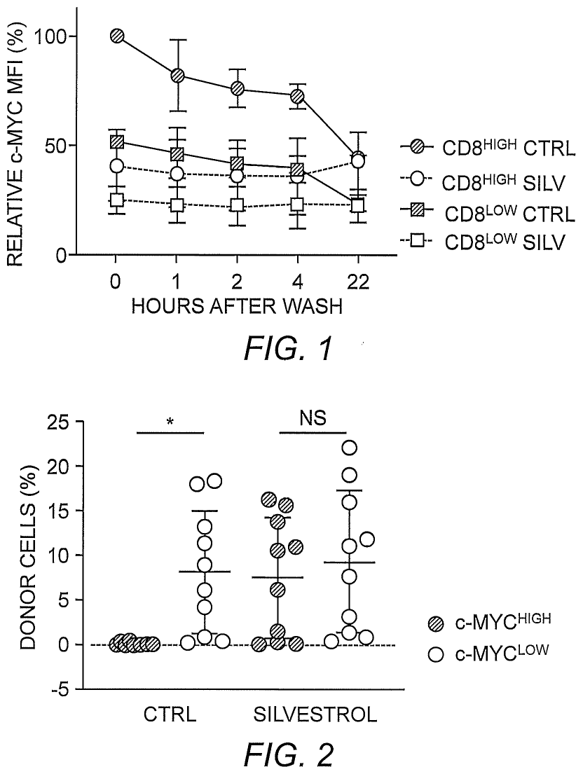

The functional consequences of eIF4A-mediated c-Myc asymmetry was analyzed in vivo. Sorted, first-division c-Myc^{high} and c-Myc^{low} cells were treated with silvestrol for 2 hours, sufficient time to reduce c-Myc levels in c-Myc^{high} cells to those observed in c-Myc^{low} cells (FIG. 1). Next, cells were washed and transferred into congenically distinct recipients. Host animals were infected with a sub-lethal dose of influenza A virus (HKx31-OVA, H3N2) before rechallenging with a heterosubtypic influenza A virus (PR8-OVA, H1N1) four weeks later. Recovery and analysis of splenic CD8⁺ T cells revealed that the sorted, untreated c-Myc^{high} cells failed to respond to the secondary infection, as previously observed previously (Verbist et al. (2016) Nature 532:389-393). However, c-Myc^{high} cells briefly treated with silvestrol were detected at numbers comparable to those found for sorted c-Myc^{low} cells (FIG. 2). These findings indicate that when a naïve CD8⁺ T cell divides for the first time following activation, the level of c-Myc expression (and potentially other eIF4A-dependent factors) dictate distinct trajectories of cellular function and furthermore, brief inhibition of eIF4A-dependent translation immediately after the first division is sufficient to alter this cell fate trajectory.

### Example 5: TORC1 Signaling is Localized at the Proximal Pole of Activated, Undivided CD8⁺ T Cells Regulating Asymmetric c-Myc Synthesis

Translation initiation is regulated by multiple signaling pathways, including the phosphoinositide 3-kinase (PI3K)-mechanistic target of rapamycin (mTOR) complex 1 (TORC1) pathway (see Roux & Topisirovic (2018) Mol. Cell Biol. 38(12):e00070-18). TORC1 phosphorylates the scaffold protein eIF4GI at serine 1108, although the functional consequences of this modification remain unclear (Raught et al. (2000) EMBO J. 19:434-444). It was determined that TORC1-mediated phosphorylation of eIF4GI was polarized both in vitro and in vivo in activated, undivided cells, and was asymmetrically distributed in dividing sister cells. Interestingly, phosphorylation of 4EBP1, a well characterized TORC-mediated modification of the translation initiation machinery, was not polarized. Like eIF4A, unphosphorylated eIF4G was neither polarized before cell division nor asymmetric after cell division, suggesting that the polarization of c-myc translation occurs via the modification of specific components of the translation initiation complex by TORC1 activity. The absence of a significant positive correlation between the distribution of phosphorylated eIF4GI and RPL26 argues against a significant contribution of uneven cytosolic volumes to the observed proximal polarization of p-eIF4GI.

This indicated that if TORC1 activity were polarized to the region of the MTOC, the local action of TORC1 would promote the synthesis of c-Myc at this pole of the cell. The spatial distribution of TORC1 activity was therefore analyzed in activated CD8⁺ T cells prior to the first division in lymph node sections from immunized animals. Active mTOR, as measured by phosphorylation of serine 2481, was polarized toward the proximal pole of the cell. Similarly, polarization of TORC1 activity to the MTOC was observed in undivided CD8⁺ T cells that were activated with plate-bound anti-CD3, anti-CD28 and ICAM1 in vitro. In contrast, non-phosphorylated mTOR showed no polarization toward the proximal pole in activated T cells. The polarization of TORC1 activity appeared to be dependent on TCR engagement, as activation of CD8⁺ T cells with phorbol myristate acetyate (PMA) plus Ionomycin, although resulting in a high level of phospho-mTOR, failed to induce its polarization toward the proximal pole. This finding is consistent with previous studies in which activation of CD8⁺ T cells with PMA plus Ionomycin was associated with symmetric distribution of TORC1 activity in dividing sister cells, and also recapitulated the phenotype of CD8^{high} and c-Myc^{high} cell populations in vivo; that is, such cells showed a reduced potential to respond to a secondary infection (Pollizzi et al. (2016) Nat. Immunol. 17:704-711).

### Example 6: Dynein-Mediated Vesicular Transport Mediates Polarization of TORC1-eIF4F Toward the Proximal Pole of Activated, Undivided CD8⁺ T Cells

The mTOR signaling pathway integrates environmental cues to direct the production of proteins, lipids, nucleotides, and ATP during cell growth (see Liu & Sabatini (2020) Nature Rev. Mol. Cell Biol. 21:183-203). The activation of TORC1 involves the recruitment of mTOR from the cytoplasm to the lysosome. In general, lysosomes are transported through the cell along microtubules (Matteoni & Kreis (1987) J. Cell Biol. 105:1253-1265). In CD8⁺ T cells, major rearrangements of the cytoskeleton occur in response to activation, including the relocalization of the MTOC to the immune synapse, to allow for directed transport of cargo, such as lysosomes, throughout the cell (Geiger et al. (1982) J. Cell Biol. 95:137-143; Ryser et al. (1982) J. Immunol. 128:1159-1162; Stinchcombe et al. (2006) Nature 443:462-465). The dynein motor proteins mediate minus-end-directed transport toward the MTOC, whereas kinesins mediate plus-end-directed transport, away from the MTOC (see Sweeney & Holzbaur (2018) Cold Spring Harb. Perspect. Biol. 10:a021931).

To explore the possibility that upon activation, lysosomal transport towards the MTOC at the proximal pole of the cell is involved in the polarization of TORC1-eIF4F signaling and subsequent c-Myc synthesis, nanoscale-resolution expansion microscopy (Zhang et al. (2020) Curr. Protoc. Neurosci. 92:e96) was employed. Shortly after activation, phosphorylated eIF4GI was observed co-localized with small vATPase structures (likely lysosomes) that arrayed along microtubules at the proximal pole of the cell. This observation was confirmed by expansion microscopy. Further, it was found in immunoprecipitation experiments that eIF4GI and eIF4A interacted with the TORC1 components Raptor and RagC and were associated with ribosomal proteins and c-myc mRNA. Brief inhibition of mTOR activity during activation was sufficient to reduce the interaction of eIF4A both with RagC and eIF4GI. Treatment of activated, undivided cells with dynein inhibitors Dynarrestin (Hoing et al. (2018) Cell. Chem. Biol. 25:357-369) or Ciliobrevin D (Firestone et al. (2012) Nature 484:125-129) abrogated the polarization of cytosolic c-Myc, as well as p-eIF4GI and TORC1. Importantly, TORC1 activity in general, as measured by phosphorylation of ribosomal protein S6, remained unaffected.

Taken together, these findings provide evidence that upon T cell activation components of the translation initiation machinery are modified by TORC1 on lysosomes, which are then transported by dynein along microtubules towards the MTOC, ultimately resulting in polarization of TORC1eIF4F signaling and polarization of c-Myc synthesis.

### Example 7: After a Single Cell Division, Sister Cells are Often Transcriptionally Distinct

It was next determined whether asymmetric synthesis of c-Myc results in transcriptomic differences between sister cells after a single cell division, indicative of distinct cell fate trajectories. An endogenous barcode transgenic mouse model (BCM) (Gerlach et al. (2013) Science 340:635-639) was employed to determine the transcriptional profiles of sister CD8⁺ T cells after a single division and assess the contribution of asymmetric cell division to early transcriptional diversity. In this system, transient V(D)J recombinase expression during T cell development results in recombination and nucleotide diversification of a pseudo-V(D)J substrate. Successful recombination drives GFP expression and provides the cell with a unique genetic barcode, which is inherited by the daughter cells upon division. This allowed the identification of individual sister cell pairs and analysis of their transcriptional profiles.

Using single-cell sequencing of barcode and gene expression libraries, 62 barcode pairs were identified from first-division CD8⁺ T cells. Once visualized with UMAP plots, it became clear that many putative sister cells could be characterized by transcriptional similarity within the pair, as depicted by two connected cells in close proximity in UMAP space. However, within another subset of sister cells, the two cells of a pair were transcriptionally distinct, as might be expected had asymmetric cell division occurred.

To investigate potential regulators of transcriptional similarity and dissimilarity between confirmed sister cell pairs in an unbiased manner, it was determined whether variation in known gene sets correlated with broadly defined signals of general transcriptional variation. Using the first two principal components from PCA analysis, Euclidean distance was calculated between putative sisters and correlations between those distances and absolute differences in gene set expression modules were identified. Of the 54 gene sets tested, significant correlations were found between sister-pair distances and gene set differences for 9 sets after adjustment for multiple comparison. These significant correlations included gene sets associated with c-Myc target gene expression, mTORC1 signaling, and a gene set previously associated with asymmetrical division and cell fate determination (Kakaradov et al. (2017) Nature Immunol. 18:422-432).

Using a probabilistic model of V(D)J rearrangement specifically for this BCM system (Marcou et al. (2018) Nat. Commun. 9:561), probabilities of generation (Peen) for observed barcodes were estimated. By comparing the barcode P_{gen} for each putative sister pair to their pairwise Euclidean PCA distance, it was found that the barcodes for transcriptionally distinct sister pairs were no more or less likely to arise in the system than those from transcriptionally similar sisters, ruling out the possibility that these analyses were biased due to potential convergence in barcode recombination. To further contextualize the extent of transcriptional differences between putative sisters, sister-pair distances were also related to the general transcriptional heterogeneity within each experiment, dividing each distance by the median distance from all randomly selected cell-cell pairs within the experiment. With this Median Random Pair Distance (MRPD) ratio, a value of 0 therefore indicates identical cell pairs, while a value of 1 indicates sisters that exhibit the median transcriptional differences between randomly selected cell pairs in the experiment. Distances between putative sister-cells were significantly greater than would be expected of identical cell pairs (p=7.766e-12, two-sided Wilcoxon Rank Sum test with a null hypothesis of 0), and on average reflected 1.03×(SD=0.75) the median transcriptional distance within an experiment.

To ensure that differences between sister pairs was not specific to the in vitro barcode model, single-cell expression datasets were also generated from first-division T cells without barcodes, stimulated in vivo. These data exhibited similar patterns to those described for the in vitro experiments. Taken together, these data suggest that a major sources of transcriptional variation across populations of first-division T cells seems to correlate with gradations in expression of c-Myc targets and mTORC1 signaling and, furthermore, that distinct transcriptional profiles between sister cells are established as early as the first division.

What is claimed is:

1. A method for preparing T cells for adoptive T cell therapy comprising contacting a population of activated T cells with an Eukaryotic initiation factor-4A (eIF4A) inhibitor thereby preparing the T cells for adoptive T cell therapy.

2. The method of claim 1, wherein the T cells are CD8⁺ T cells.

3. The method of claim 2, wherein activation is via stimulation of CD3, CD28, or a combination thereof.

4. The method of claim 1, further comprises expanding the activated population of T cells with one or more cytokines.

5. The method of claim 1, further comprising introducing into said population of T cells an exogenous nucleic acid molecule thereby producing a population of engineered T cells.

6. The method of claim 5, wherein the exogenous nucleic acid molecule encodes an antigen recognizing receptor, an ortho-receptor, an immunomodulatory cytokine, a chemokine receptor, a dominant-negative receptor, or a transcription factor for preventing exhaustion.

7. The method of claim 6, wherein the antigen is a tumor antigen, a self-antigen, or a pathogen antigen.

8. The method of claim 6, wherein said antigen recognizing receptor is a T cell receptor (TCR) or a chimeric antigen receptor (CAR).

9. A population of T cells prepared by the method of claim 1.

10. A pharmaceutical composition comprising the population of T cells of claim 9 in admixture with a pharmaceutically acceptable carrier or vehicle.

11. A method for adoptive T cell therapy comprising administering to a subject in need of adoptive T cell therapy the pharmaceutical composition of claim 10.

12. A population of engineered T cells prepared by the method of claim 5.

13. A pharmaceutical composition comprising the population of engineered T cells of claim 12 in admixture with a pharmaceutically acceptable carrier or vehicle.

14. A method of treating cancer comprising administering to a subject in need of treatment the pharmaceutical composition of claim 13.

15. A kit for preparing T cells for adoptive T cell therapy comprising
(a) an Eukaryotic initiation factor-4A (eIF4A) inhibitor, and
(b) a CD3 agonist.

16. The kit of claim 15, further comprising (c) one or more costimulatory ligands.

17. The kit of claim 15, further comprising (c) one or more cytokines.


SEQUENCE LISTING (11 sequences)

SEQ ID NO: 1 - protein, 8 aa, Artificial sequence
  Synthetic peptide
SIINFEKL

SEQ ID NO: 2 - DNA, 46 bp, Artificial sequence
  Synthetic oligonucleotide
aatgatacggcgaccgagatctacactctttccctacacgacgctc

SEQ ID NO: 3 - DNA, 20 bp, Artificial sequence
  Synthetic oligonucleotide
gctgaacttgtggccgttta

SEQ ID NO: 4 - DNA, 22 bp, Artificial sequence
  Synthetic oligonucleotide
aatgatacggcgaccgagatct

SEQ ID NO: 5 - DNA, 19 bp, Artificial sequence
  Synthetic oligonucleotide
cgtccagctcgaccaggat

SEQ ID NO: 6 - DNA, 17 bp, Artificial sequence
  Synthetic oligonucleotide
ttacctcctcgaggtca

SEQ ID NO: 7 - DNA, 16 bp, Artificial sequence
  Synthetic oligonucleotide
catggtgagcaagggc

SEQ ID NO: 8 - DNA, 23 bp, Artificial sequence
  Synthetic oligonucleotide
tttgtctatttggggacagtgtt

SEQ ID NO: 9 - DNA, 18 bp, Artificial sequence
  Synthetic oligonucleotide
catcgtcgtggctgtctg

SEQ ID NO: 10 - DNA, 22 bp, Artificial sequence
  Synthetic oligonucleotide
atgctaagctgtcgcacaaatg

SEQ ID NO: 11 - DNA, 20 bp, Artificial sequence
  Synthetic oligonucleotide
gttcgatccgtaaccgatgt